(19) United States
(12) Patent Application Publication
(10) Pub. No.: US 2024/0261295 A1
(43) Pub. Date: Aug. 8, 2024

(54) COMBINATIONS OF WEE1 INHIBITORS AND ANTI-CD47 ANTIBODIES

(71) Applicant: Recurium IP Holdings, LLC, San Diego, CA (US)

(72) Inventors: Fernando Donate, Santa Monica, CA (US); Hooman Izadi, San Diego, CA (US); Ahmed Abdi Samatar, West Windsor, NJ (US); Brant Clayton Boren, San Diego, CA (US); Kevin Duane Bunker, Escondido, CA (US); Peter Qinhua Huang, San Diego, CA (US)

(21) Appl. No.: 18/634,055

(22) Filed: Apr. 12, 2024

Related U.S. Application Data
(63) Continuation of application No. PCT/US22/46291, filed on Oct. 11, 2022.
(60) Provisional application No. 63/262,466, filed on Oct. 13, 2021.

(51) Int. Cl. A61K 31/5365; A61K 31/519; A61K 39/00; A61P 35/00; C07K 16/28

(52) U.S. Cl. CPC A61K 31/5365; A61K 31/519; A61P 35/00; C07K 16/2803; A61K 2039/505

(57) ABSTRACT

Disclosed herein are combinations of compounds for treating a disease or condition, such as cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/262,466, filed Oct. 13, 2021, which is incorporated by reference in its entirety. The present application is a continuation of PCT Application No. PCT/US2022/046291, filed Oct. 11, 2022, which claims priority to U.S. Provisional Application No. 63/262,466, filed Oct. 13, 2021, each of which is hereby incorporated by reference in their entireties.

### FIELD

The present application relates to the fields of chemistry, biochemistry and medicine. More particularly, disclosed herein are combination therapies, and methods of treating diseases and/or conditions with combination therapies described herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2021, roughly 1.9 million people will be diagnosed with cancer, and an estimated 600,000 people will die of cancer in the United States. Thus, there still exists a need for effective cancer treatments.

### SUMMARY

Some embodiments described herein relate to a combination of compounds that can include an effective amount of Compound (A), or a pharmaceutically acceptable salt thereof, and an effective amount of Compound (B), or a pharmaceutically acceptable salt of any of the foregoing.

Some embodiments described herein relate to the use of a combination of compounds for treating a disease or condition, wherein the combination includes an effective amount of Compound (A), or a pharmaceutically acceptable salt thereof, and an effective amount of Compound (B), or a pharmaceutically acceptable salt of any of the foregoing. Other embodiments described herein relate to the use of a combination of compounds in the manufacture of a medicament for treating a disease or condition, wherein the combination includes an effective amount of Compound (A), or a pharmaceutically acceptable salt thereof, and an effective amount of Compound (B), or a pharmaceutically acceptable salt of any of the foregoing.

In some embodiments, the disease or condition can be a cancer described herein.

### DRAWINGS

FIG. 1 provides structures of WEE1 inhibitors.

FIG. 2 shows the effect of using a WEE1 inhibitor, or a pharmaceutically acceptable salt thereof, and an anti-CD47 antibody alone or in combination on tumor volume in a RPMI-8226 non-Hodgkin lymphoma xenograft model.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. Those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to the use of a combination of compounds for treating a disease or condition, wherein the combination can include an effective amount of Compound (A), or a pharmaceutically acceptable salt thereof, and an effective amount of Compound (B), or a pharmaceutically acceptable salt of any of the foregoing, wherein Compound (A) is a WEE1 inhibitor; and Compound (B) is an anti-CD47 antibody.

Examples of suitable WEE1 inhibitors for Compound (A) include those described in the following publications: WO 2019/074979, WO 2020/210383, WO 2020/210375, WO 2020/210377, WO 2020/210380, WO 2020/210381, WO 2022/082174, U.S. 2022/0162229, U.S. 2022/0168313, U.S. 2022/0169646, U.S. 2022/0220115, U.S. Pat. No. 11,332,473, WO 2019/173082, WO 2019/011228, WO 2019/138227, WO 2018/162932, WO 2018/011570, WO 2018/011569, US 2022/0194947, WO 2018/090939, WO 2015/092431, WO 2015/019037, WO 2014/167347, WO 2007/126122, WO 2011/034743, U.S. 2007/0254892, WO 2008/133866, U.S. 2016/0060258, U.S. 2019/0308984, U.S. 2020/0131192, WO 2021/073491, U.S. Pat. Nos. 11,345,710, 11,345,711 WO 2019/085933, WO 2020/221358, EP 3712150, WO 2018/133829, WO 2021/047627, US 2021/0403451, WO 2020/083404, WO 2019/037678, WO 2018/171633, CN 113387962, WO 2019/165204, WO 2012/161812, WO 2013/012681, WO 2013/013031, WO 2013/059485, WO 2013/126656, U.S. 2012/0220572, U.S. 2013/0018045, KR 2016035878, KR 2020016567, WO 2018/056621, WO 2017/075629, WO 2019/169065, WO 2019/134539, WO 2020/028814, US 2021/0309630, WO 2020/069105, WO 2020/192581, U.S. 2022/0194960, CN 114831993, CN 111718348, WO 2022/188802, WO 96/34867, WO 2008/153207, WO 2010/067888, WO 2009/054332, WO 2021/073491, WO 2021/074251, CN 112142763, WO 2020/259724, U.S. 2022/0259210, WO 2019/096322, CN 112142747, CN 112142747, WO 2021/043152, WO2021/254389, WO 2022/171088, WO 2022/171126, WO 2022/171128, WO 2022/174765, WO 2022/174796, CN 112442049, CN 114072411, CN 113402520, CN113387962, KR 2022081171, WO 2022/124748, WO 2022/155202, CN 114591334 and WO 2021/074251.

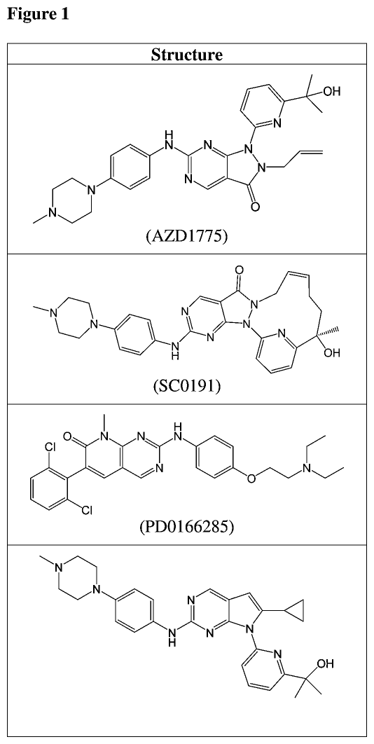

In some embodiments, the WEE1 inhibitor can be selected from AZD1775, SC0191, PD0166285, NUV-569, SDR-7995, SDR-7778, IMP7068, Debio 0123, SY-4835, SPH-6162 and ATRN-W1051, or any combination thereof. Further details regarding WEE1 inhibitors are provided in FIG. 1. In other embodiments, the WEE1 inhibitor can be

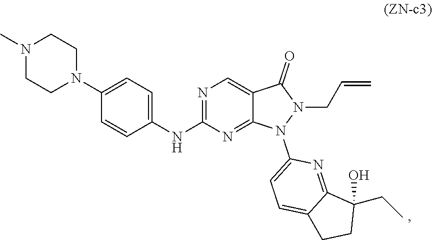 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof. In still other embodiments, the WEE1 inhibitor can be

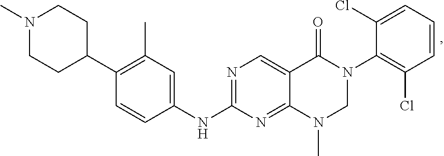 [SMILES: Cc1cc(Nc2ncc3c(n2)N(C)CN(c2c(Cl)cccc2Cl)C3=O)ccc1C1CCN(C)CC1]

or a pharmaceutically acceptable salt or N-oxide thereof. In yet still other embodiments, the WEE1 inhibitor can be selected from

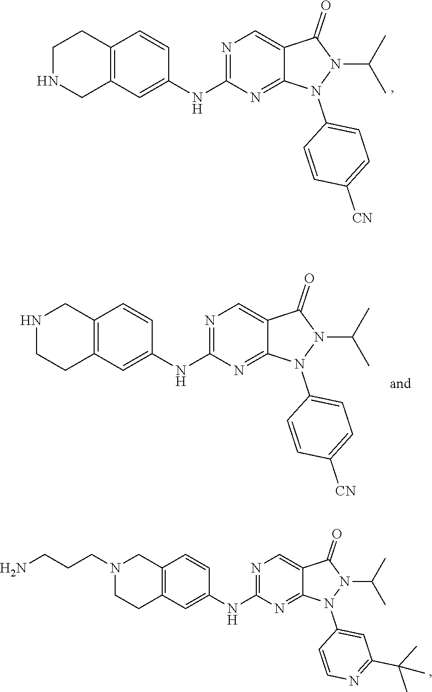

or a pharmaceutically acceptable salt thereof of any of the foregoing. In some embodiments, the WEE1 inhibitor can be selected from

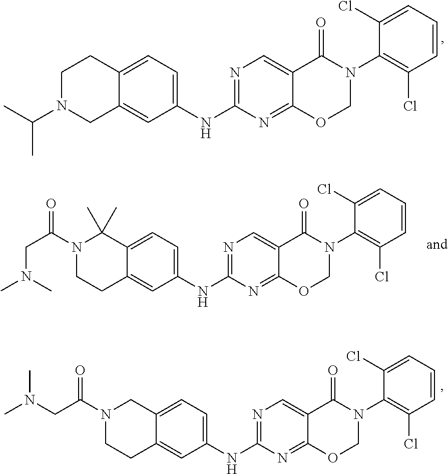 [SMILES: CC(C)N1CCc2ccc(Nc3ncc4c(n3)OCN(c3c(Cl)cccc3Cl)C4=O)cc2C1 ; CN(C)CC(=O)N1CCc2cc(Nc3ncc4c(n3)OCN(c3c(Cl)cccc3Cl)C4=O)ccc2C1(C)C ; CN(C)CC(=O)N1CCc2cc(Nc3ncc4c(n3)OCN(c3c(Cl)cccc3Cl)C4=O)ccc2C1]

or a pharmaceutically acceptable salt thereof of any of the foregoing. In other embodiments, the WEE1 inhibitor can be

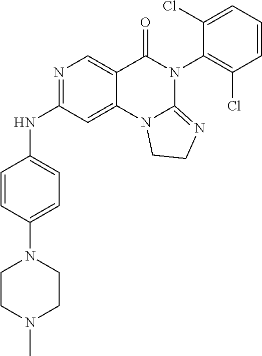 [SMILES: CN1CCN(c2ccc(Nc3cc4c(cn3)C(=O)N(c3c(Cl)cccc3Cl)C3=NCCN34)cc2)CC1]

or a pharmaceutically acceptable salt thereof. In still other embodiments, the WEE1 inhibitor can be selected from

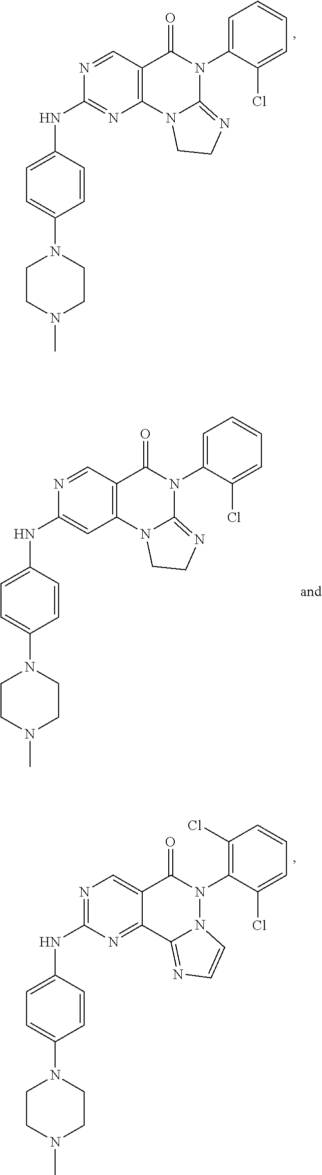 [SMILES: CN1CCN(c2ccc(Nc3ncc4c(n3)N3CCN=C3N(c3ccccc3Cl)C4=O)cc2)CC1 ; CN1CCN(c2ccc(Nc3cc4c(cn3)C(=O)N(c3ccccc3Cl)C3=NCCN34)cc2)CC1 ; CN1CCN(c2ccc(Nc3ncc4c(=O)n(-c5c(Cl)cccc5Cl)n5ccnc5c4n3)cc2)CC1]

or a pharmaceutically acceptable salt thereof of any of the foregoing.

Exemplary anti-CD47 antibodies include the following: STI-6643 (Sorrento therapeutics), Magrolimab (Gilead), DSP107 (KAHR), IBI-188 (Innovent Biologics), AK117 (Akeso), ALX148 (ALX Oncology), AO-176 (Arch Oncology), HX009 (Waterstone Hanxbio), lemzoparlimab (Abbvie/I-Mab), SRF231 (Surface Oncology), ZL-1201 (Zai Lab) and TTI-621 (Pfizer).

The order of administration of compounds in a combination described herein can vary. In some embodiments, Compound (A), including pharmaceutically acceptable salts thereof, can be administered prior to Compound (B), or a pharmaceutically acceptable salt thereof. In other embodiments, Compound (A), including pharmaceutically acceptable salts thereof, can be administered concomitantly with Compound (B), or a pharmaceutically acceptable salt thereof. In still other embodiments, Compound (A), including pharmaceutically acceptable salts thereof, can be administered subsequent to the administration of Compound (B), or a pharmaceutically acceptable salt thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein of Compound (A), including pharmaceutically acceptable salts thereof, and Compound (B), or pharmaceutically acceptable salts thereof, can decrease the number and/or severity of side effects that can be attributed to a compound described herein, such as Compound (B), or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts thereof, and Compound (B), or pharmaceutically acceptable salts thereof, can result in an additive effect. In some embodiments, a combination as described herein of Compound (A), including pharmaceutically acceptable salts thereof, and Compound (B), or pharmaceutically acceptable salts thereof, can result in a synergistic effect. In some embodiments, a combination as described herein of Compound (A), including pharmaceutically acceptable salts thereof, and Compound (B), or pharmaceutically acceptable salts thereof, can result in a strongly synergistic effect. In some embodiments, a combination as described herein of Compound (A), including pharmaceutically acceptable salts thereof, and Compound (B), or pharmaceutically acceptable salts thereof, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts thereof, can be provided in a pharmaceutical composition. Likewise, Compound (B), including pharmaceutically acceptable salts thereof, can be provided in a pharmaceutical composition.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as antioxidants and metal-chelating agents are excipients. In an embodiment, the pharmaceutical composition comprises an antioxidant and/or a metal-chelating agent. A “diluent” is a type of excipient.

In some embodiments, Compounds (B), along with pharmaceutically acceptable salts thereof, can be provided in a pharmaceutical composition that includes Compound (A), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts thereof, can be administered orally. In some embodiments, Compound (A), including pharmaceutically acceptable salts thereof, can be provided to a subject by the same route of administration as Compound (B), along with pharmaceutically acceptable salts thereof. In other embodiments, Compound (A), including pharmaceutically acceptable salts thereof, can be provided to a subject by a different route of administration as Compound (B), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A), including pharmaceutically acceptable salts thereof, and an effective amount of Compound (B), or a pharmaceutically acceptable salt of any of the foregoing, can be used to treat a disease or condition.

In some embodiments, the disease or condition can be selected from glioblastoma, astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer (such as colon adenocarcinoma), endometrial cancer, esophagus cancer, eye cancer, gallbladder cancer, gastric cancer, gastrointestinal cancer, Hodgkin lymphoma, Non-Hodgkin lymphoma, hematological tumor, Kaposi sarcoma, kidney cancer, laryngeal and hypopharyngeal cancer, liver cancer, lung cancer, non-small cell lung cancer, small cell, lung cancer, lymphoma, mesothelioma, melanoma, multiple myeloma, neuroblastoma, nasopharyngeal cancer, ovarian cancer, osteosarcoma, sarcomas, gastrointestinal stromal tumor (GIST), pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor and a liquid tumor. In some embodiments, the cancer can be non-Hodgkin lymphoma. In some embodiments, the cancer can be a colorectal cancer (for example, colon adenocarcinoma).

In some cases, following cancer treatment, a subject can relapse or have reoccurrence of the cancer. As used herein, the terms “relapse” and “reoccurrence” are used in their normal sense as understood by those skilled in the art. Thus, the cancer can be a recurrent cancer.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of Compounds (A) and/or (B), or pharmaceutically acceptable salts of the foregoing, can be determined by comparing their in vitro activity, and in vivo activity in animal models. Such comparison can be done by comparison against an established drug, such as cisplatin and/or gemcitabine.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Examples

Additional embodiments are disclosed in further detail in the following examples, which are not in any way intended to limit the scope of the claims.

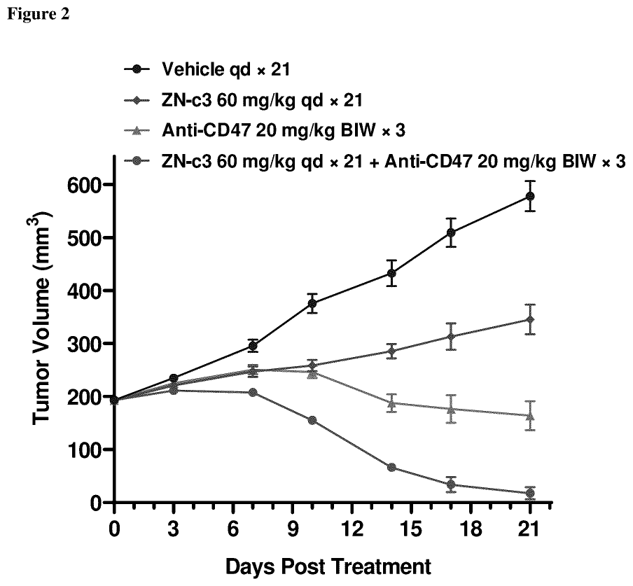

Mice were inoculated with RPMI-8226 cells subcutaneously on the right flank with the single cell suspension of 95% viable tumor cells (1×107) in 100 μL RPMI 1640 Matrigel mixture (1:1 ratio) without serum for the tumor development. The treatment was started when the mean tumor size reached approximately 200 mm³, with individual tumor size ranging from 185-235 mm³. Animals were randomly distributed into treatment groups of 10 animals each and dosed with vehicle and indicated compounds at the indicated dosage and frequency shown in FIG. 2 and Table 1. In FIG. 2, the bottom line with circles is ZN-c3 60 mg/kg p.o. qd×21+Anti-CD47 20 mg/kg ip BIW×3. Anti-CD47 was obtained from Bio X Cell (clone B6.H12, catalog number BE0019-1). Tumor volumes were evaluated twice per week to calculate tumor volume over time, and the mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as individual tumor volume (TV) decrease (terminal TV compared to initial TV). The percent tumor regression was calculated using the formula: (1−(Td/T0))×100%. FIG. 2 and Table 1 illustrate that single agent treatment of ZN-c3 at 60 mg/kg resulted in tumor growth inhibition of 61% and single agent treatment with Anti-CD47 20 mg/kg ip BIW×3 resulted in 108% efficacy and 15% regression. The combination of ZN-c3 (60 mg/kg) and Anti-CD47 20 mg/kg ip BIW×3 exhibited significant TGI and resulted in the complete regression of 8 out of 10 tumors on day 21.

TABLE 1

| | | Tumor | Complete |
|---|---|---|---|
| | TGI % | Regression % | Regression |
| Compound | (Day 21) | (Day 21) | (Day 21) |
| ZN-c3 (60 mg/kg) | 61 | — | — |
| Anti-CD47 (20 mg/kg) | 108 | 15 | 0/10 |
| ZN-c3 (60 mg/kg) + | 109 | 91 | 8/10 |
| Anti-CD47 | | | |
| (20 mg/kg) | | | |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

What is claimed is:

1. Use of a combination of compounds for treating a disease or condition, wherein the combination includes an effective amount of Compound (A) and an effective amount of Compound (B), or a pharmaceutically acceptable salt of any of the foregoing, wherein Compound (A) is a WEE1 inhibitor; and Compound (B) is an anti-CD47 antibody.

2. The use of claim 1, wherein the WEE1 inhibitor is provided in any one or more of the following publications: WO 2019/074979, WO 2020/210383, WO 2020/210375, WO 2020/210377, WO 2020/210380, WO 2020/210381, WO 2022/082174, U.S. 2022/0162229, U.S. 2022/0168313, U.S. 2022/0169646, U.S. 2022/0220115, U.S. Pat. No. 11,332,473, WO 2019/173082, WO 2019/011228, WO 2019/138227, WO 2018/162932, WO 2018/011570, WO 2018/011569, US 2022/0194947, WO 2018/090939, WO 2015/092431, WO 2015/019037, WO 2014/167347, WO 2007/126122, WO 2011/034743, U.S. 2007/0254892, WO 2008/133866, U.S. 2016/0060258, U.S. 2019/0308984, U.S. 2020/0131192, WO 2021/073491, U.S. Pat. Nos. 11,345,710, 11,345,711 WO 2019/085933, WO 2020/221358, EP 3712150, WO 2018/133829, WO 2021/047627, US 2021/0403451, WO 2020/083404, WO 2019/037678, WO 2018/171633, CN 113387962, WO 2019/165204, WO 2012/161812, WO 2013/012681, WO 2013/013031, WO 2013/059485, WO 2013/126656, U.S. 2012/0220572, U.S. 2013/0018045, KR 2016035878, KR 2020016567, WO 2018/056621, WO 2017/075629, WO 2019/169065, WO 2019/134539, WO 2020/028814, US 2021/0309630, WO 2020/069105, WO 2020/192581, U.S. 2022/0194960, CN 114831993, CN 111718348, WO 2022/188802, WO 96/34867, WO 2008/153207, WO 2010/067888, WO 2009/054332, WO 2021/073491, WO 2021/074251, CN 112142763, WO 2020/259724, U.S. 2022/0259210, WO 2019/096322, CN 112142747, CN 112142747, WO 2021/043152, WO2021/254389, WO 2022/171088, WO 2022/171126, WO 2022/171128, WO 2022/174765, WO 2022/174796, CN 112442049, CN 114072411, CN 113402520, CN113387962, KR 2022081171, WO 2022/124748, WO 2022/155202, CN 114591334 and WO 2021/074251.

3. The use of claim 1 or 2, wherein the WEE1 inhibitor is selected from the group consisting of AZD1775, SC0191, PD0166285, NUV-569, SDR-7995, SDR-7778, IMP7068, Debio 0123, SY-4835, SPH-6162 and ATRN-W1051, or any combination thereof.

4. The use of any one of claims 1-3, wherein the WEE1 inhibitor is 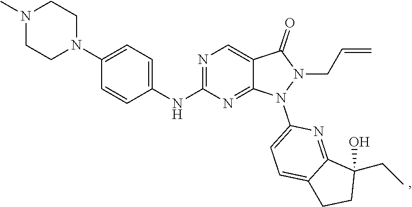 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof.

5. The use of any one of claims 1-3, wherein the WEE1 inhibitor is 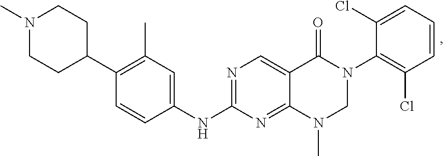 [SMILES: Cc1cc(Nc2ncc3c(n2)N(C)CN(c2c(Cl)cccc2Cl)C3=O)ccc1C1CCN(C)CC1]
or a pharmaceutically acceptable salt or N-oxide thereof.

6. The use of any one of claims 1-3, wherein the WEE1 inhibitor is selected from the group consisting of: 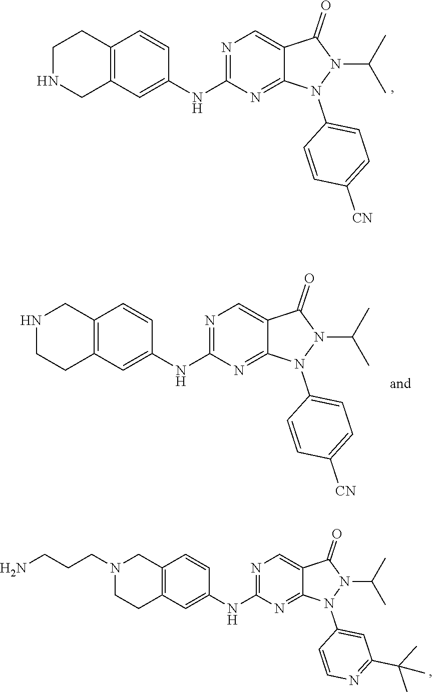
or a pharmaceutically acceptable salt thereof of any of the foregoing.

7. The use of any one of claims 1-3, wherein the WEE1 inhibitor is selected from the group consisting of: 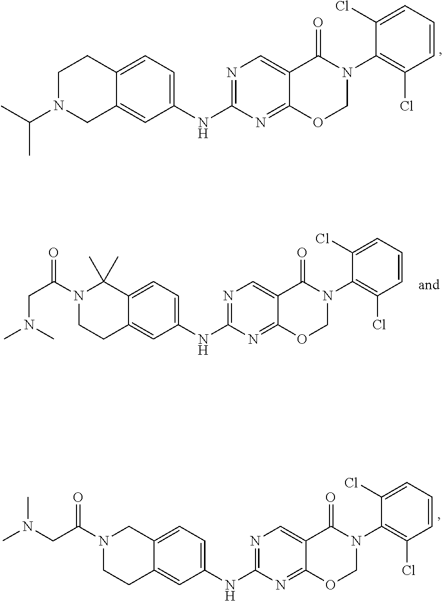 [SMILES: CC(C)N1CCc2ccc(Nc3ncc4c(n3)OCN(c3c(Cl)cccc3Cl)C4=O)cc2C1 ; CN(C)CC(=O)N1CCc2cc(Nc3ncc4c(n3)OCN(c3c(Cl)cccc3Cl)C4=O)ccc2C1(C)C ; CN(C)CC(=O)N1CCc2cc(Nc3ncc4c(n3)OCN(c3c(Cl)cccc3Cl)C4=O)ccc2C1]
or a pharmaceutically acceptable salt thereof of any of the foregoing.

8. The use of any one of claims 1-3, wherein the WEE1 inhibitor is 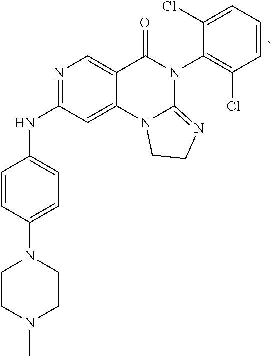 [SMILES: CN1CCN(c2ccc(Nc3cc4c(cn3)C(=O)N(c3c(Cl)cccc3Cl)C3=NCCN34)cc2)CC1]
or a pharmaceutically acceptable salt thereof.

9. The use of any one of claims 1-3, wherein the WEE1 inhibitor is selected from the group consisting of: 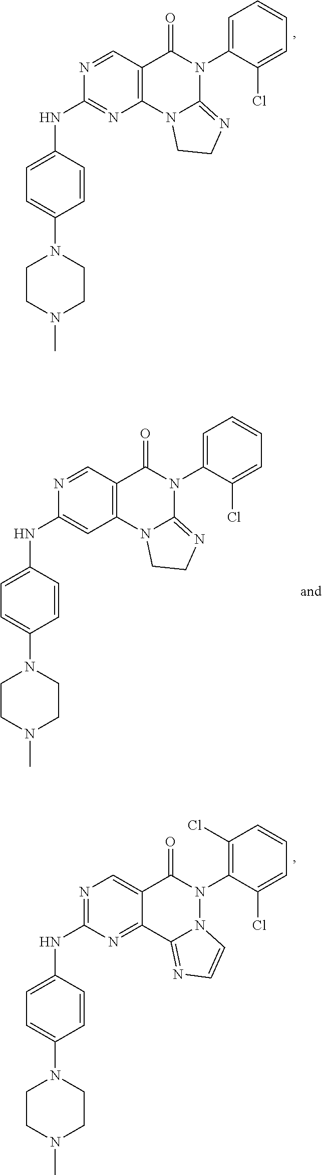 [SMILES: CN1CCN(c2ccc(Nc3ncc4c(n3)N3CCN=C3N(c3ccccc3Cl)C4=O)cc2)CC1 ; CN1CCN(c2ccc(Nc3cc4c(cn3)C(=O)N(c3ccccc3Cl)C3=NCCN34)cc2)CC1 ; CN1CCN(c2ccc(Nc3ncc4c(=O)n(-c5c(Cl)cccc5Cl)n5ccnc5c4n3)cc2)CC1]
or a pharmaceutically acceptable salt thereof of any of the foregoing.

10. The use of any one of claims 1-9, wherein the anti-CD47 antibody is selected from the group consisting of STI-6643 (Sorrento therapeutics), Magrolimab (Gilead), DSP107 (KAHR), IBI-188 (Innovent Biologics), AK117 (Akeso), ALX148 (ALX Oncology), AO-176 (Arch Oncology), HX009 (Waterstone Hanxbio), lemzoparlimab (Abbvie/I-Mab), SRF231 (Surface Oncology), ZL-1201 (Zai Lab) and TTI-621 (Pfizer).

11. The use of any one of claims 1-10, wherein the disease or condition is selected from the group consisting of glioblastoma, astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastric cancer, gastrointestinal cancer, Hodgkin lymphoma, Non-Hodgkin lymphoma, hematological tumor, Kaposi sarcoma, kidney cancer, laryngeal and hypopharyngeal cancer, liver cancer, lung cancer, non-small cell lung cancer, small cell, lung cancer, lymphoma, mesothelioma, melanoma, multiple myeloma, neuroblastoma, nasopharyngeal cancer, ovarian cancer, osteosarcoma, sarcomas, gastrointestinal stromal tumor (GIST), pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor and a liquid tumor.

12. The use of any one of claims 1-10, wherein the disease or condition is non-Hodgkin lymphoma.
